(19) United States
(12) Patent Application Publication
(10) Pub. No.: US 2007/0162138 A1
(43) Pub. Date: Jul. 12, 2007

(54) Vertebral implant and insertion tool

(75) Inventor: Eric Steven Heinz, Memphis, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/299,580

(22) Filed: Dec. 12, 2005

(51) Int. Cl. A61F 2/44

(52) U.S. Cl. 623/17.16

(57) ABSTRACT

A vertebral implant and insertion tool for placing implants in the spine are disclosed. The implant has a tool engaging surface configured for intimate engagement with the insertion tool. The insertion tool configuration is particularly suited for being gripped at a plurality of angles. The insertion tool having a gripping end adapted for intimate engagement with an implant at a plurality of angles.

### FIELD OF THE INVENTION

The present invention is directed to improved implants, implant inserters, and methods of their use. More particularly, in one aspect the present invention is directed to spinal implants and instrumentation for use in performing spinal surgery.

### BACKGROUND OF THE INVENTION

The present invention relates to implants and instruments for inserting implants into the skeletal system. This may have particular application to the human spine. A number of medical conditions such as compression of spinal cord nerve roots, degenerative disc disease, herniated nucleus pulposis, spinal stenosis, and spondylolisthesis can cause severe low back pain. Restoration of the space between adjacent vertebrae and/or removal of the anatomical structure pushing against the spinal cord or exiting nerve roots is known to alleviate patient suffering. Some intervertebral implants rest on the existing endplates while others either partially or completely extend into the adjacent intervertebral bodies. Access to the affected disc space is sought from a variety of approaches and angles to the spine. The approach and angle chosen depends upon surgeon preference, patient anatomy, level of the spine affected, and interbody implant selection.

Therefore, there remains a need for improved implant designs, configurations of the tool engagement surface on the implant, as well as improvement of the insertion tools utilized to grasp the implant during the insertion procedure.

### SUMMARY OF THE INVENTION

The present invention provides an implant for positioning at least partially between two vertebrae. The implant comprises an implant body having an upper engaging surface for engaging at least a portion of an upper vertebral body and an opposite lower engaging surface for engaging at least a portion of a lower vertebral body. A tool engagement configuration is formed on the implant body. The tool engagement configuration is adapted for engagement by an insertion tool over a range of angles with respect to a longitudinal axis of the insertion tool. The tool engagement configuration includes an upper gripping surface and a lower gripping surface. In one aspect, the implant is annular. In a further aspect, the implant includes a plurality of tool engagement configurations.

In another aspect, the present invention provides an insertion tool for holding an implant adapted for insertion at least partially into the skeletal system. The insertion tool comprises a shaft having a length and an implant gripping end. A longitudinal axis extends along at least a portion of the length of the shaft. The implant gripping end is adapted to receive an implant at a range of angles with respect to the longitudinal axis. In one aspect, the insertion tool also includes an actuator adapted for moving the implant gripping end between an open position and a locked position for selectively engaging the implant.

In another aspect, the present invention provides a combination implant for insertion at least partially between two vertebrae and an insertion tool for gripping the implant at various angles. The combination comprises an implant body having a first engagement area and an insertion tool. The insertion tool includes a shaft having a length and an implant gripping end. The implant gripping end is located at a distal end of the shaft and configured for gripping the implant at a plurality of angles via the first engagement area.

Further aspects, forms, embodiments, objects, features, benefits, and advantages of the present invention shall become apparent from the detailed drawings and descriptions provided herein.

### BRIEF DESCRIPTION OF THE DRAWINGS

FIG. 1A is a top view of an implant according to one embodiment of the present invention.

FIG. 1B is a partial cross-sectional view of the implant of FIG. 1 taken along section line 1B-1B.

FIG. 2 is a perspective view of the implant of FIG. 1 in combination with an implant insertion tool being positioned into a disc space between adjacent vertebrae according to one embodiment of the present invention.

FIG. 3 is a perspective view of the implant of FIG. 1 engaged with a portion of an implant insertion tool according to one embodiment of the present invention.

FIG. 4A is an enlarged partial side view of the implant insertion tool of FIG. 2.

FIG. 4B is an enlarged partial perspective view of the implant insertion tool of FIG. 2.

FIG. 4C is an enlarged partial cross-sectional view taken along section line 4C-4C.

FIG. 5A is a partial side view of the implant and insertion tool gripping end shown in substantial alignment with a horizontal plane.

FIG. 5B is a partial side view of the implant and insertion tool combination shown in FIG. 5A, shown in a first offset position.

FIG. 5C is a partial side view of the implant and insertion tool combination shown in FIG. 5A, shown in a second offset position.

FIG. 6A is a top view of the implant of FIG. 1 partially engaged with an inserter in a third offset position according to one embodiment of the present invention.

FIG. 6B is a top view of the implant of FIG. 1 partially engaged with an inserter in a fourth offset position according to one embodiment of the present invention.

FIG. 7A is a partial perspective view of a further embodiment of an inserter according to the present invention.

FIG. 7B is a partial cross-sectional view of an alternative embodiment of an implant according to the present invention.

FIG. 8A is a partial cross-sectional view of an alternative embodiment of an implant according to the present invention.

FIG. 8B is a partial cross-sectional view of an alternative embodiment of an implant according to the present invention.

FIG. 8C is a partial cross-sectional view of an alternative embodiment of an implant according to the present invention.

FIG. 9 is a partial cross-sectional view of an alternative embodiment of an implant according to the present invention.

FIG. 10 is a top view of an implant according to one embodiment of the present invention.

### DESCRIPTION OF THE PREFERRED EMBODIMENTS

For the purposes of promoting an understanding of the principles of the present invention, reference will now be made to the embodiments illustrated in the drawings, and specific language will be used to describe the same. It will nevertheless be understood that no limitation of the scope of the invention is intended thereby. Any alterations and further modifications in the described devices, instruments, methods, and any further application of the principles of the invention as described herein are contemplated as would normally occur to one skilled in the art to which the invention relates.

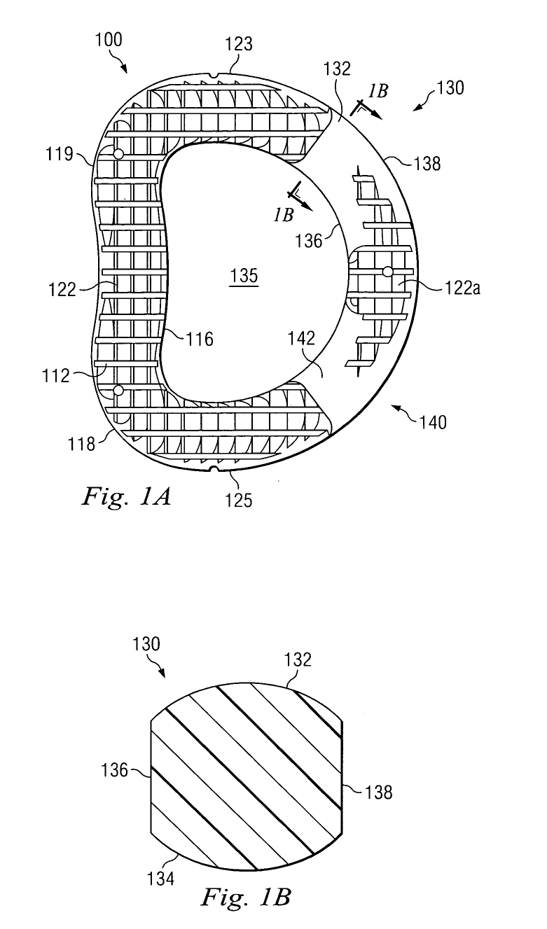

Referring now to FIG. 1A, there is shown a top view of an annular implant 100 according to one aspect of the present invention. As shown more fully in FIGS. 1A-3, the implant 100 includes an upper surface 112 and an opposing lower surface 114; each configured for contact with and/or placement in close approximation to the bone of adjacent upper and lower vertebrae, respectively. Further, at least a portion of the upper and lower surfaces 112, 114 are load bearing surfaces. The upper surface 112 includes a plurality of projections 122. In a similar manner, the lower surface 114 includes a plurality of projections 124. The projections 122 and 124 are adapted for engaging bone to maintain the relative position of the implant 100 in the disc space between adjacent vertebrae. In the illustrated embodiment, an interior sidewall 116 extends between the upper and lower surfaces 112, 114 and defines an internal cavity 135. An exterior sidewall 118 extends between the upper and lower surfaces 112, 114. In the illustrated embodiment, the exterior sidewall 118 has a posterior portion 119, an opposing anterior portion 121, a first side portion 123, and an opposite second side portion 125. The configuration of the exterior sidewall 118 may be adapted to form implants of various shapes that either singularly or in combination are designed to extend between two vertebrae.

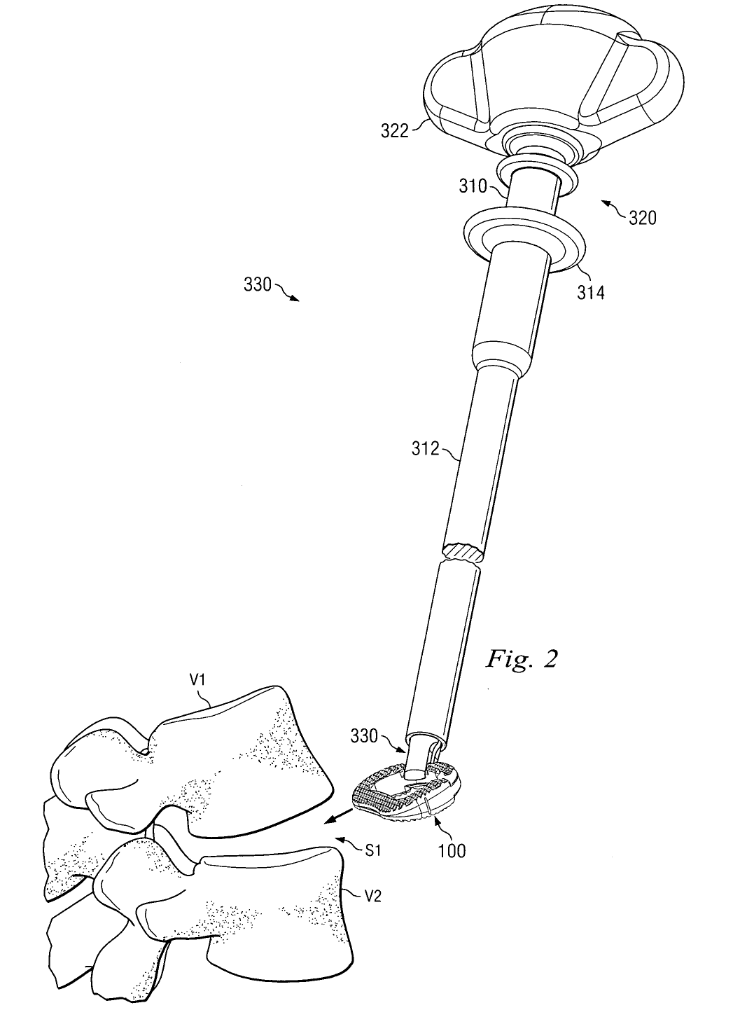

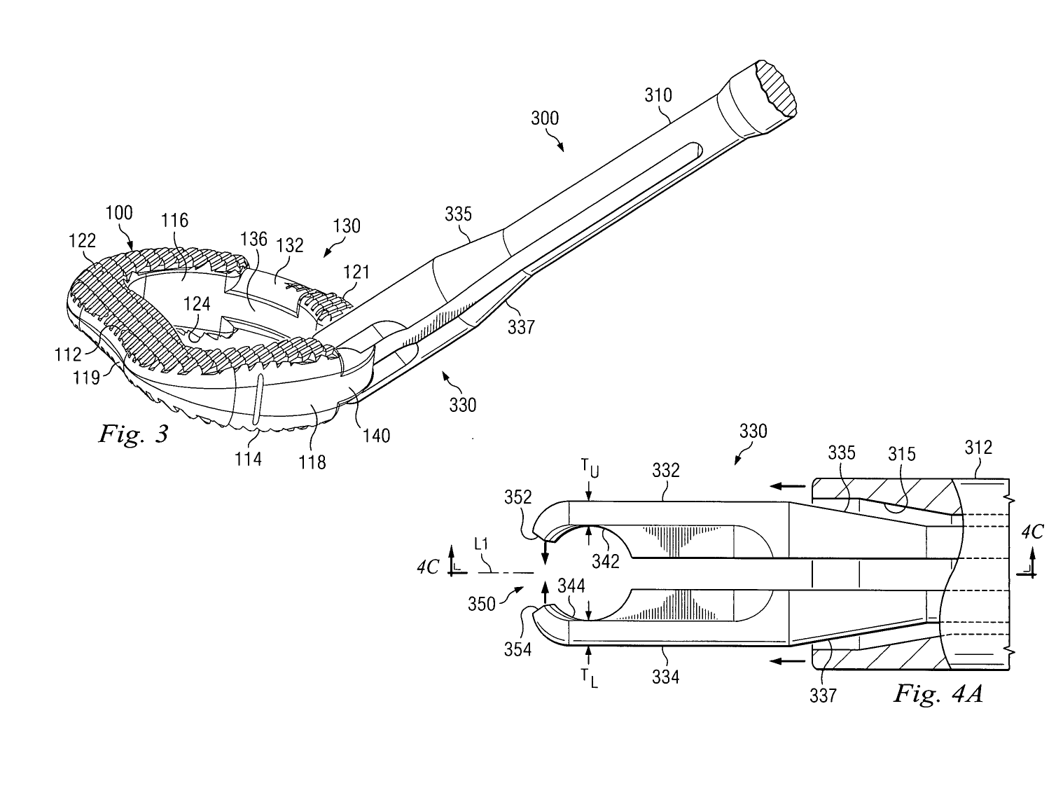

Referring to FIGS. 2 and 3, it is contemplated that the height of the implant 100 will be selected to substantially maintain the height of an at least partially restored disc space S1 between two adjacent vertebrae V1 and V2. It will be appreciated that the height of the implant 100 can be configured to substantially match the naturally occurring surfaces of the vertebral endplate and spacing of a restored height. As illustrated in FIG. 3, to assist in maintaining natural lordosis, the height of the exterior sidewall 118 increases from the posterior portion 119 to the anterior portion 121. Alternatively, the height of the implant 100 may be formed to match a disc space prepared to a desired space by bone removed from one or both of the endplates adjacent to disc space.

The implant 100 includes two tool engagement configurations 130, 140 spaced by projections 122(a) on the top surface 112. The tool engagement configuration 130 includes an upper gripping surface 132 and a lower gripping surface 134. The upper and lower gripping surfaces 132, 134 are substantially convex. As shown in FIG. 1B, the gripping surfaces 132, 134 are uniformly convex surfaces having a substantially identical but opposed radii of curvature in cross section. In the illustrated embodiment, the radius of curvature generates an arc having a diameter greater than the height of the implant. In the illustrated embodiment, an interior sidewall 136 and an exterior sidewall 138 interrupt the upper and lower arcuate gripping surfaces 132, 134 and each sidewall has a height extending between the upper and lower gripping surfaces. Further, the height of the interior and exterior sidewalls 136, 138 is less than the adjacent interior and exterior sidewalls 116, 118 such that the upper and lower gripping surfaces 132, 134 are recessed with respect to the upper and lower surfaces 112, 114. Further, it should be appreciated that the interior and exterior sidewalls 136, 138 are arcuate in a horizontal plane of the implant, as shown in FIG. 1A, to match the annular design of the implant 100. That is, the interior sidewall 136 is concave in the horizontal plane, while the exterior sidewall 138 is convex in the horizontal plane. Thus, tool engagement configuration 130 has an arcuate shape in the horizontal plane of the implant.

The orientation of the upper and lower gripping surfaces 132, 134 to the interior and exterior sidewalls 136, 138 forms the partially cylindrical shaped tool engagement configuration 130, as better seen in the cross-sectional view of FIG. 1B. Tool engagement configuration 140 has similar construction. While the interior and exterior sidewalls 136, 138 of the tool engagement configuration are shown as being flat in a vertical plane of the implant, it is fully contemplated that the sidewalls may have an arcuate shape between the upper and lower gripping surfaces 132, 134. In the illustrated embodiment, the upper and lower gripping surfaces 132, 134 have a smooth texture. It is contemplated, however, that the upper and lower gripping surfaces 132, 134 will be grit blasted, shot peened, grooved, knurled, roughened, chemically etched, or otherwise configured to encourage gripping. In the current embodiment, it is understood that the tool engagement configurations 140 is substantially similar to the tool engagement configuration 130. FIG. 3 shows the implant 100 engaged with an implant insertion tool 300 via the tool engagement configuration 140.

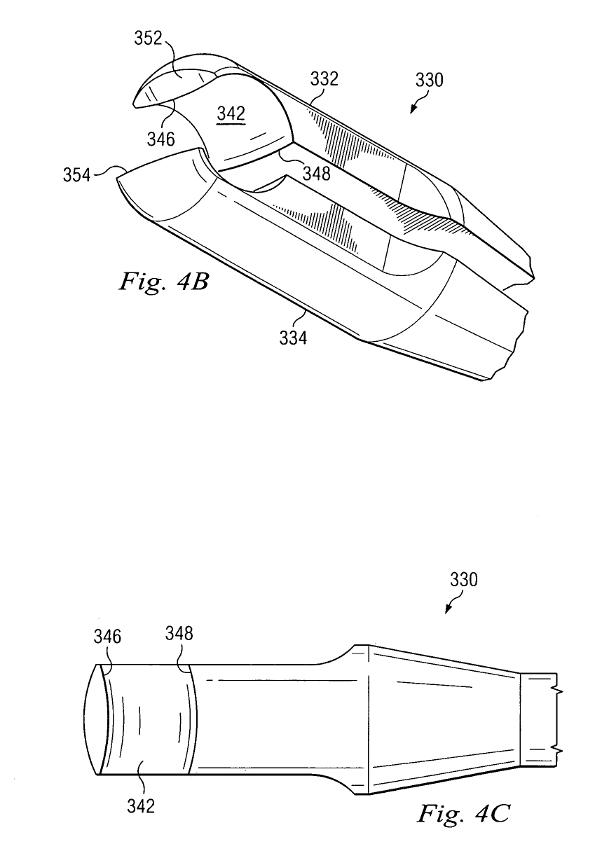

Referring now to FIG. 2, there is shown a multi-grip insertion tool 300 in combination with an implant 100 in accordance with another aspect of the present invention. As shown more fully in FIGS. 3-4B, the insertion tool 300 includes an elongated shaft 310, an actuator end 320, and an implant gripping end 330. The elongated shaft 310 extends along a longitudinal axis L1. The shaft 310 may be bifurcated so that the shaft itself forms the implant gripping end 330. The insertion tool 300 further includes an elongated outer tube 312 slidably coupled with the elongated shaft 310. The outer tube 312 includes an inner surface defining an internal tapered shoulder 315. The inner shaft 310 includes a pair of external opposing tapered surfaces 335, 337 configured to engage the internal tapered shoulder 315 of the outer tube 312 upon longitudinal movement along longitudinal axis L1 towards the implant gripping end 330 and, thereby, move the gripping end to a closed position or locked position. The actuator end 320 acts to move the outer tube 312 along the longitudinal axis L1 between an open and closed position for selectively engaging the implant gripping end 330. In the illustrated embodiment, the outer tube 312 is biased by a spring (not shown) to urge the outer tube to a closed or locked position. Thus, the implant gripping end 330 is selectively disengaged by moving the outer tube 312 to an open position. This is accomplished by pulling an outer tube lip 314 along the longitudinal axis L1 towards a handle 322 located adjacent to the actuator end 320 of the insertion tool 300. Releasing the lip 314 will cause the springs (not shown) to return the outer tube 312 down the longitudinal axis L1 to an engaged position.

FIG. 4A shows the implant gripping end 330 where the outer tube 312 is being held in an open position. The implant gripping end 330 includes an upper portion 332 having a thickness T_{U} and a lower portion 334 having a thickness T_{L}. In the illustrated embodiment, the upper and lower portions 332, 334 are bifurcated portions of the shaft 310 configured so that the upper and lower portions are moveable with respect to each other and are naturally biased to the open position. The upper portion 332 includes a substantially concave inner surface 342 configured for engaging an implant at a plurality of angles, as better illustrated in FIG. 4B. The lower portion 334 also includes a substantially concave inner surface 344 configured for engaging an implant at a plurality of angles. The orientation of inner surfaces 342, 344 to one another in cross section creates a partially cylindrical channel 350 for receiving an implant 100 at a plurality of angles with respect to the longitudinal axis L1. In the current embodiment, the inner surfaces 342, 344 are configured to mate with the substantially convex upper and lower gripping surfaces 132, 134 of the tool engagement configuration 130. This allows the insertion tool 300 to grip the implant 100 over a continuous range of angles with respect to the longitudinal axis L1.

Further, since the upper and lower gripping surfaces 132, 134 are recessed with respect to the upper and lower surfaces 112, 114, the thicknesses T_{U}, T_{L} of the upper and lower portions 332, 334 are substantially thin so as to minimize the invasiveness of the engaged implant-insertion tool combination when inserting the implant. Further, the radius of curvature R1 of the upper portion 332 and the radius of curvature R2 of the lower portion 334 are adapted to minimize the invasiveness of the implant-insertion tool combination. At some angles with respect the longitudinal axis L1 the thicknesses T_{U}, T_{L} of the upper and lower portions 332, 334 are such that the upper and lower portions are substantially planar with the upper and lower surfaces 112, 114. Further, at some angles with respect to the longitudinal axis L1 the upper or lower portions 332, 334 may be partially recessed with respect to the upper and lower surfaces 112, 114.

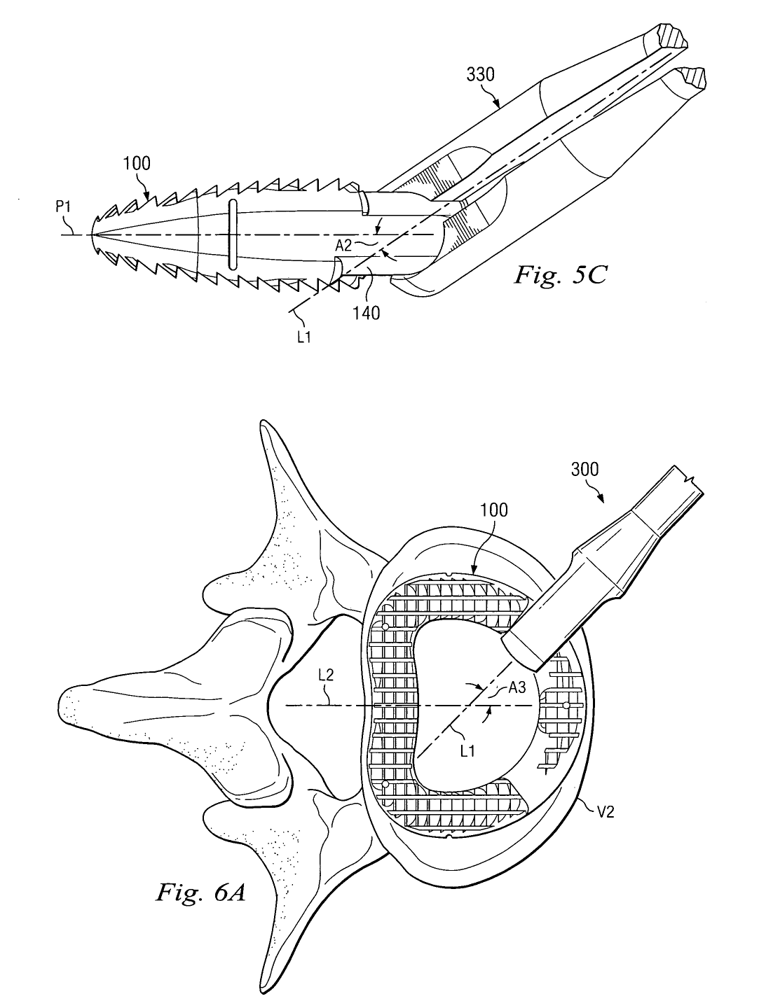

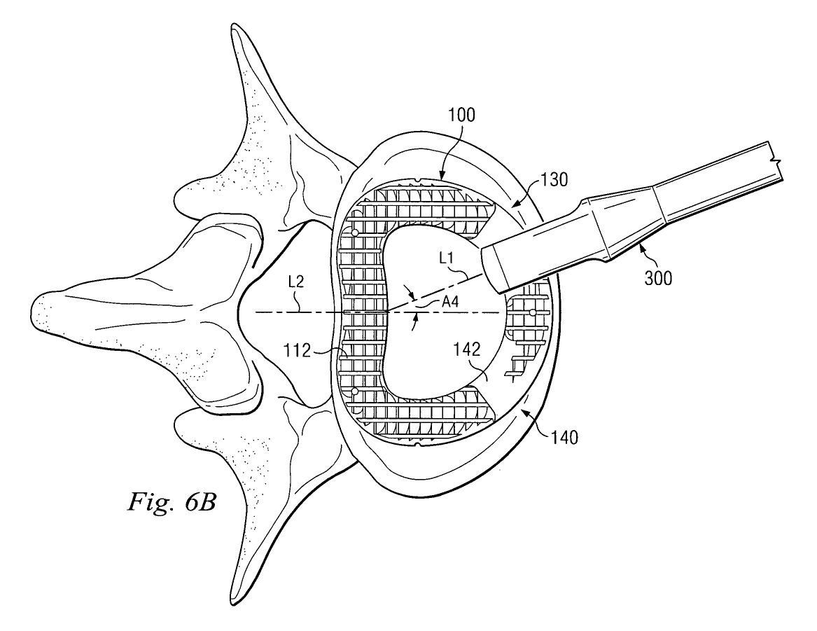

The upper and lower portions 332, 334 also have upper and lower ends 352, 354, respectively. As the outer tube 312 moves down the longitudinal axis L1 from an open to a closed position, the shoulder 315 of the outer tube engages the external surfaces 335, 337 of the shaft 310 and forces the upper and lower ends 352, 354 towards each other causing the implant gripping end 330 to securely grip the implant 100 that is at least partially located within the channel 350. The distance between the upper and lower ends 352, 354 is such that when the outer tube 312 is in an open position an implant may be inserted into or released from the channel 350. However, when the outer tube 312 is in a closed position the distance between the upper and lower ends 352, 354 is such that the implant 100 within the channel 350 is securely gripped by the insertion tool 300. It is contemplated that the outer tube 312 may also have an intermediate position where the implant 100 within the channel 350 is moveably engaged by the insertion tool 300. That is, the distance between the upper and lower ends 352, 354 is sufficiently close to prevent the implant 100 from being released by the insertion tool 300, however, the implant may be rotated within the channel 350 allowing the insertion angle to be changed. Thus, this type of intermediate position provides the ability to change the insertion angle even after the insertion has begun. Further, the intermediate position allows for translational movement as well. That is, the insertion tool 300 may slide around the annular curvature of the implant 100 for additional insertion angles, as better seen in FIGS. 6A and 6B.

Referring now to FIG. 4C, the inner gripping surface 342 includes a distal edge 346 and a proximal edge 348. In addition to the curvature of the gripping surface 342 in an upward direction away from the longitudinal axis L1, the distal edge 346 and proximal edge 348 are curved in a horizontal plane of the insertion tool to substantially match the annular curvature of the implant. Thus, distal edge 346 has a convex curvature and proximal edge 348 has a concave curvature. In the illustrated embodiment, the radius of curvature of the proximal edge 348 is greater than the radius of curvature for the distal edge 346. It is contemplated that the radius of curvature of the proximal edge 348 be substantially the same as the radius of curvature for the distal edge 346. Further, it is contemplated that the radius of curvature of the proximal edge 348 be less than the radius of curvature for the distal edge 346. The inner gripping surface 344 has similarly curved edges in the horizontal plane. This curvature of the inner gripping surfaces 342, 344 may assist the insertion tool 300 to grip the engagement configurations 130, 140 at various positions in the horizontal plane of the implant, as illustrated in FIGS. 6A and 6B.

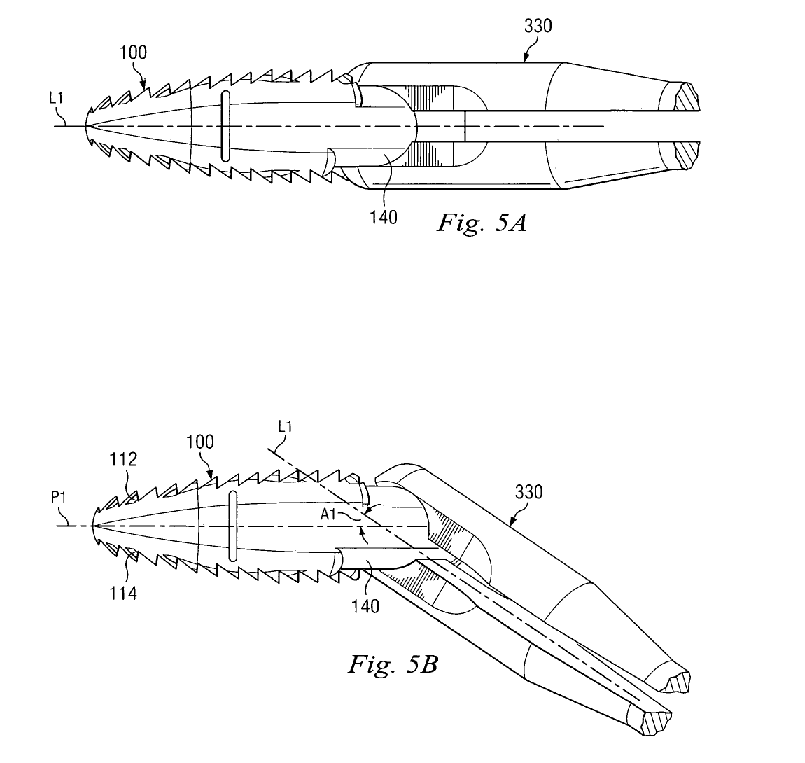

Referring now to FIGS. 5A-5C, there is shown the insertion tool 300 gripping the implant 100 at multiple angles with respect to the horizontal plane of the implant. Axis P1 extends within the horizontal plane of the implant 100 and is provided as a reference for use in describing the relative positions of the implant and the insertion tool. It will be understood that an implant axis may be extend in alternative directions with the angular position of the inserter taken with respect to this alternative implant axis. In FIG. 5A, the insertion tool 300 is gripping the implant 100 directly along the longitudinal axis L1 such that the insertion tool is in alignment with axis P1 of the implant. In FIG. 5B, the insertion tool 300 is gripping the implant 100 at an angle A1 with respect to the axis P1 such that the insertion tool is approaching the implant from below. In FIG. 5C, the insertion tool 300 is gripping the implant 100 at an oblique angle A2 with respect to the axis P1 such that the insertion tool is approaching the implant from above. FIGS. 5A-5C illustrate how the insertion tool 300 is able to grip the implant 100 via the tool engagement configuration 140 at a plurality of angles with respect to the axis P1. Though not illustrated, it is fully contemplated that the insertion tool 300 may grip the implant 100 over a continuous range of angles A1 from above to an offset angle A2 below the implant. In a preferred aspect, angles A1 and A2 are substantially equal. Angles A1 and A2 may have a maximum range from approximately 45 degrees to 5 degrees. More preferably, angles A1 and A2 may have a maximum range of approximately 30 degrees.

Referring now to FIGS. 6A and 6B, there is shown the insertion tool 300 gripping the implant 100 at different angles with respect to the anterior-to-posterior axis L2 of the implant 100. The insertion tool 300 is able to grip the tool engagement configurations 130, 140 in a plurality of positions along the implant 100 because the tool engagement channel 350 substantially follows the curvature of the implant. This provides additional alternatives for insertion angles and approaches when inserting the implant 100 with the insertion tool 300. Thus, the insertion tool 300 is configured to grip the implant 100 at a plurality of angles with respect to both the axis P1 and the anterior-to-posterior axis L2 of the implant.

In FIG. 6A, inserter 300 grips implant 100 at an angle A3 between axis L1 and axis L2. Preferably angle A3 is between approximately 60 degrees and 40 degrees, but more preferably about 45 degrees. In FIG. 6B, inserter 300 grips implant 100 at an angle A4 between axis L1 and axis L2. Preferably angle A4 is between approximately 40 degrees and 15 degrees, but more preferably about 30 degrees. As a result in the illustrated embodiments, the inserter 300 may grip the implant 100 in a range of angles over an arc of approximately 45 degrees with respect to the anterior-to-posterior axis L2 of the implant.

In operation, the implant gripping end 330 of the inserter and/or the tool engagement configuration 130 allow a surgeon to engage the inserter to the implant in a variety of orientations permitting optimal insertion angles. In one surgical technique, the insertion tool 300 and the implant 100 may be utilized as follows. First, if necessary, perform a standard block discectomy and decompression. Next, utilize a trial with the insertion tool 300 in order to determine the proper implant height and/or correction angle. Finally, insert the implant 100 with the insertion device 300. Referring now to FIG. 2, the implant 100 is shown engaged with insertion tool 300 and extending into the disc space S1 adjacent vertebra V1 through an opening in the annulus. It will be appreciated, that the illustrated technique demonstrates an anterior approach to the disc space from an oblique angle relative to the anterior to posterior axis. While the illustrated embodiment of inserter and implant have been shown with reference to utilization in this type of technique, the present invention is not limited to a particular approach to the spine and the teachings and principles of the present invention may be utilized for other surgical techniques. For example, the implant 100 may be configured as an implant suitable for a TLIF, PLIF, or lateral procedure with the gripping surfaces oriented to allow engagement by an inserter used in these approaches. Moreover, the inserter may be used with a variety of spacers, either implants or instruments, including but not limited to, bone grafts, spacers, distractors, trials, corpectomy devices, nucleus replacements, artificial discs, facet replacements, or any other implant.

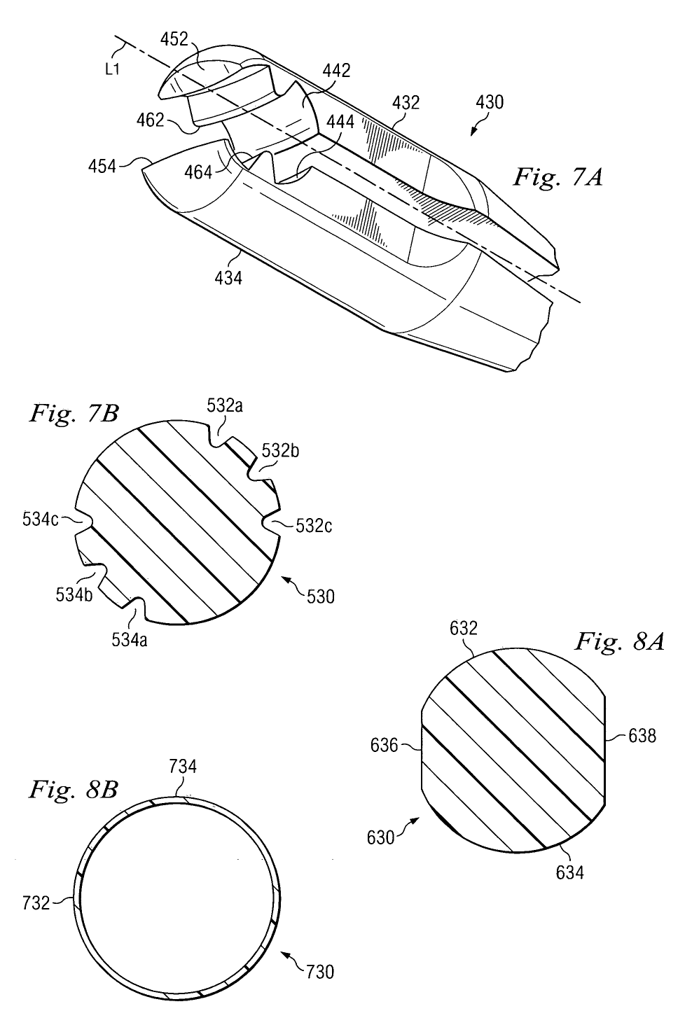

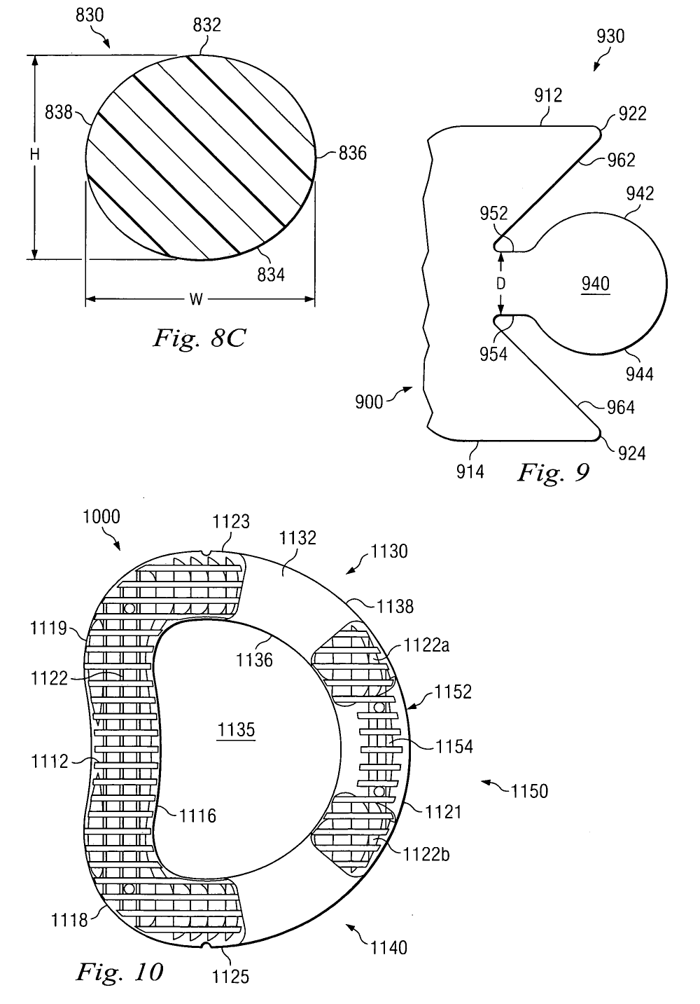

Referring now to FIGS. 7A and 7B, there is shown an alternative embodiment of an implant inserter 400. Inserter 400 has many similarities to insertion tool 300 above. For example, inserter 400 includes an elongated shaft (not shown), an actuator end (not shown), and an implant gripping end 430. The implant gripping end 430 includes an upper portion 432 and a lower portion 434. In the illustrated embodiment, the upper and lower portions 432, 434 are moveable with respect to each other for selectively engaging an implant. The actuator end moves the upper and lower portions 432, 434 between open and closed positions for selectively engaging the implant. The upper portion 432 includes a substantially concave inner surface 442. The upper inner surface 442 includes a convex projection 462 configured for engaging a cavity of an implant. The lower portion 434 also includes a substantially concave inner surface 444 configured for engaging a cavity of an implant. Further, the lower inner surface 444 includes a convex projection 464 for engaging a cavity of an implant. For example, as shown in FIG. 8B in the partial cross-sectional view of a tool engagement configuration 530 of an implant, the upper and lower convex projections 462, 464 are configured to engage three upper cavities 532a, 532b, and 532c and three lower cavities 534a, 534b, and 534c, respectively. The choice of multiple engagement cavities provides the inserter 400 with the means to grab the implant at a plurality of discrete angles with respect to a longitudinal axis L1 of the inserter. It is fully contemplated that the tool engagement configuration 530 may include more or less engagement cavities to allow for insertion at a plurality of discrete angles with respect to the longitudinal axis L1. Further, it is contemplated that the each projection 462, 464 may be adapted to engage a single cavity at a plurality of angles.

FIGS. 8A-8C each illustrate partial cross-sectional views of alternative embodiments of an implant according other aspects of the present invention. In particular, FIGS. 8A-8C each represent cross-sectional views of a tool engagement configuration similar to the cross-sectional views of the tool engagement configurations 130, 530 in FIGS. 1B and 7B, respectively. It is fully contemplated that the implants illustrated in FIGS. 8A-8C are substantially similar to the implant 100 described above.

FIG. 8A shows a partial cross-sectional view of a tool engagement configuration 630 of an implant similar to implant 100 of FIG. 1A. The tool engagement configuration 630 is configured for engagement by an inserter at a plurality of angles with respect to a longitudinal axis of the inserter. The tool engagement configuration 630 includes upper and lower gripping surfaces 632, 634 and interior and exterior sidewalls 636, 638. As illustrated, the exterior sidewall 638 has a vertical height greater than the height of the interior sidewall 636. However, it is also fully contemplated that interior sidewall 636 may have a height greater than the height of the exterior sidewall 638.

FIG. 8B shows a partial cross-sectional view of a tool engagement configuration 730 of an implant similar to implant 100 of FIG. 1A. The tool engagement configuration 730 is configured for engagement by an inserter at a plurality of angles with respect to a longitudinal axis of the inserter. The tool engagement configuration 730 includes a substantially cylindrical surface 732. The cylindrical surface is shown as being grit blasted surface and extending to a solid core 734. The gripping surface 732 is grit blasted to help facilitate gripping of the implant 700 by an insertion tool. It is fully contemplated that the gripping surface 732 may be otherwise textured or configured to encourage gripping of the implant 700 via the tool engagement configuration 730.

FIG. 8C shows a partial cross-sectional view of a tool engagement configuration 830 of an implant similar to implant 100 of FIG. 1A. The tool engagement configuration 830 is configured for engagement by an inserter at a plurality of angles with respect to a longitudinal axis of the inserter. The tool engagement configuration 830 has arcuate upper and lower walls 832, 834, respectively, defining a width W. Upper and lower walls 832, 834 are spaced apart by arcuate sidewalls 836, 838 defining a height H. The tool engagement configuration 830 is oblong such that the height H is less than the width W. It is fully contemplated, however, that the width W may be less than the height H.

FIG. 9 shows a partial cross-sectional view of an implant 900 according to another aspect of the present invention. The implant 900 includes an upper bone engaging surface 912 and an opposing lower bone engaging surface 914; each configured for contact with and/or placement in close approximation to the bone of adjacent upper and lower vertebrae, respectively. The upper and lower surfaces 912, 914 are load bearing surfaces. Further, the upper and lower surfaces 912, 914 have outer edges 922, 924, respectively. The implant 900 also includes a tool engagement configuration 930.

The tool engagement configuration 930 is configured for engagement by an inserter at a plurality of angles with respect to a longitudinal axis of the inserter. For example, the tool engagement configuration 930 may be configured for multi-angle engagement by an inserter similar to insertion tool 300 described and illustrated above. For simplicity and without limitation to the availability of alternative inserters, the implant and tool engagement configuration 930 will be explained as being configured for use with the insertion tool 300 and references will be made to the reference numerals found in FIGS. 2-4B.

The tool engagement configuration 930 includes a projection 940. The projection 940 extends beyond outer edges 922, 924 and, therefore, extends beyond the perimeter of the upper and lower surfaces 912, 914. The projection 940 has a substantially cylindrical shape with upper and lower gripping surfaces 942, 944. The upper and lower gripping surfaces 942, 944 are substantially convex. The upper and lower gripping surfaces 942, 944 are adapted to substantially mate with the concave inner surfaces 342, 344 of the insertion tool 300 to allow engagement at a plurality of angles. The projection 940 also includes upper and lower stop surfaces 952, 954 having a distance D between them. The upper and lower stop surfaces 952, 954 limit the range of angles that the insertion tool 300 may engage the implant 900. As the insertion tool 300 travels through engagement angles above the longitudinal axis L1 the upper end 352 of the insertion tool will approach and eventually hit the upper stop surface 952 when the upper limit of the angle range is reached. Similarly, as the insertion tool 300 travels through engagement angles below longitudinal axis L1 the lower end 354 of the insertion tool will approach and eventually hit the lower stop surface 954 when the lower limit of the angle range is reached. Thus, the range of engagement angles is inversely proportional to the distance D between the upper and lower stop surfaces 952, 954: the greater the distance D, the smaller the range of available engagement angles; the smaller the distance D, the greater the range of available engagement angles.

The tool engagement configuration 930 also includes upper and lower exterior surfaces 962, 964. In the illustrated embodiment the upper and lower exterior surfaces 962, 964 are angled in from the upper and lower edges 922, 924, respectively. Thus, the exterior surfaces 962, 964 also serve to limit the range of engagement angles. In the illustrated embodiment, the exterior surfaces 962, 964 limit the range of engagement angles to approximately 45 degrees above or below the longitudinal axis L1 of the insertion tool 300. However, it is fully contemplated that the upper and lower exterior surfaces 962, 964 may be angled outwardly or extended directly between edges 922, 924 so as to not restrict the range of engagement angles any more than the upper and lower stop surfaces 952, 954.

With respect to tool engagement configuration 930, it is contemplated that alternative corresponding shapes may be utilized for the protrusion 140 and the insertion tool 300 to allow for engagement at multiple angles. For example, a ball and socket structure may be utilized, where the protrusion 140 is substantially spherical and the insertion tool 300 is adapted for engaging the protrusion at multiple angles that may include angles within the horizontal plane of the implant and/or oblique to the horizontal plane of the implant. Further, the orientation of the projection and recess may be switched between the tool engagement configuration 930 and the insertion tool 300. That is, the insertion tool 300 may have a projection with convex surfaces adapted for engaging a concave recess of the tool engagement configuration 930 at a plurality of angles. In such an alternative, it is contemplated that the projection of the insertion tool may be selectively expanded or protruded for selectively engaging the concave recesses of the implant 900. Further, while singular projections and recesses have been disclosed in this embodiment, it is contemplated that multiple recesses and projections may be utilized to provide additional insertion angles.

Referring now to FIG. 10, there is shown a top view of a spacer 1000 according to one aspect of the present invention. The spacer 1000 includes an upper surface 1112 and an opposing lower surface (not shown); each configured for contact with and/or placement in close approximation to the bone of an upper and a lower vertebrae, respectively. Further, at least a portion of the upper surface 1112 and lower surface are load bearing surfaces. The upper surface 1112 includes a plurality of projections 1122. In a similar manner, the lower surface includes a plurality of projections (not shown). The projections are adapted for engaging bone to maintain the relative position of the spacer 1000 between vertebrae. In the illustrated embodiment, an interior sidewall 1116 extends between the upper surface 1112 and the lower surface and defines an internal cavity 1135. An exterior sidewall 1118 extends between the upper surface 1112 and the lower surface. In the illustrated embodiment, the exterior sidewall 1118 has a posterior portion 1119, an opposing anterior portion 1121, a first side portion 1123, and an opposite second side portion 1125. The configuration of the exterior sidewall 1118 may be adapted to form spacers of various shapes that either singularly or in combination are designed to extend between two vertebrae.

The spacer 1000 includes three tool engagement configurations 1130, 1140, and 1150 spaced by projections 1122(a) and 1122(b) on the top surface 1112. The tool engagement configuration 1130 includes an upper gripping surface 1132 and a lower gripping surface (not shown). The upper gripping surface 1132 and the lower gripping surface are substantially convex. In the illustrated embodiment, an interior sidewall 1136 and an exterior sidewall 1138 interrupt the upper and lower arcuate gripping surfaces and each sidewall has a height extending between the upper and lower gripping surfaces. Further, the height of the interior and exterior sidewalls 1136, 1138 is less than the adjacent interior and exterior sidewalls 1116, 1118 such that the upper gripping surface and the lower gripping surface are recessed with respect to the upper and lower surfaces, respectively. Further, it should be appreciated that the interior and exterior sidewalls 1136, 1138 are arcuate in a horizontal plane of the spacer to match the annular design of the spacer 1000. That is, the interior sidewall 1136 is concave in the horizontal plane, while the exterior sidewall 1138 is convex in the horizontal plane. Thus, tool engagement configuration 1130 has an arcuate shape in the horizontal plane of the spacer.

The orientation of the upper and lower gripping surfaces 1132, 1134 to the interior and exterior sidewalls 1136, 1138 forms the partially cylindrical shaped tool engagement configuration 1130. It is understood that tool engagement configurations 1140, 1150 have similar construction. In the illustrated embodiment, however, the upper gripping surface 1152 of tool engagement configuration 1150 includes a plurality of projections 1154. Similarly, the lower surface (not shown) of tool engagement configuration 1150 includes a plurality of projections (not shown). The projections of the upper and lower surfaces of tool engagement configuration 1150 are adapted for engaging bone. However, the projections do not inhibit the ability of the spacer 1000 to be engaged at a plurality of angles via tool engagement configuration 1150 because the tool engagement configuration 1150 is at least partially cylindrical in shape. In the illustrated embodiment, the upper gripping surfaces of tool engagement configurations 1130, 1140 have a smooth texture. It is contemplated, however, that the upper and lower gripping surfaces of tool engagement configurations 1130, 1140, 1150 will be grit blasted, shot peened, grooved, knurled, roughened, chemically etched, or otherwise configured to encourage gripping.

The implants described above may be formed of any material suitable for implantation. Such implants may include prostheses used to preserve motion in the disc space and those designed for rigid stabilization. Further, implant 100 may be a trial or distractor used to evaluate the fit in the disc space before final insertion of a permanent device. The insertion tools described above are generally formed of medical grade materials suitable for such applications, including stainless steel and titanium. In one aspect, the implant may be formed of a material that is softer or more brittle than the material of the inserter such that the implant may at least partially yield to the gripping force applied by the gripping end of the inserter. For example, the inserter may be formed of stainless steel and the implant formed of cortical bone. Alternatively, the implant may be formed of a resorbable polymer, such as PLDLA or similar compounds. While not exhaustive and without limitation to the use of other implant materials, examples include: stainless steel, titanium, PEEK, polymers, hydroxyapetite, biphasic calcium, coral, ceramic compounds, composite bone, allograft, autograft and xenograft.

The implants 100, 900, and 1000 described above have been illustrated as having either one, two, or three tool engagement configurations. This description has been made without limitation and for the purposes of illustration only, it being fully contemplated that the implants may have any number of tool engagement configurations. In a similar manner, the location of the tool engagement configurations has been for the purpose of illustration only and is not intended to limit the placement or position at other locations on the implant. Further, it is fully contemplated that the entire exterior surface of the implant may serve as a tool engagement configuration, allowing for engagement by an inserter over a plurality of angles with respect to the inserter at any point on the implant. Also, though the implants have been described as being annular or ring shaped, it is fully contemplated that the implants may be substantially solid and have substantially linear or arcuate exterior configurations instead.

While in some instances singular projections and recesses have been disclosed, it is contemplated that multiple recesses and projections may be utilized. Further, in other instances multiple protrusions and cavities have been disclosed, it is contemplated, however, that a single protrusion and cavity may be utilized.

The multi-grip insertion tools 300 and 400 described above have been illustrated as having implant gripping ends with concave inner surfaces. This description has been made without limitation and for the purposes of illustration only. It is fully contemplated that the implant gripping ends may have convex inner surfaces for engaging an implant over a plurality of angles. Further, the implant gripping ends may have a plurality of inner or outer surfaces adapted for engaging an implant over a plurality of angles; each surface having a particular shape and/or texture to encourage engagement of the implant.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

What is claimed is:

1. A spacer for positioning at least partially between two vertebrae, comprising:
an spacer body having an upper bone engaging surface for engaging at least a portion of an upper vertebral body, an opposite lower bone engaging surface for engaging at least a portion of a lower vertebral body, and an spacer axis extending in a horizontal plane located between the upper bone engaging surface and lower bone engaging surface; and
a first tool engagement configuration formed on the spacer body, wherein the tool engagement configuration includes an upper gripping surface and a lower gripping surface, and wherein the first tool engagement configuration is adapted for engagement by an insertion tool at a plurality of angular orientations with respect to the spacer axis.

2. The spacer of claim 1, wherein the spacer is annular.

3. The spacer of claim 1, wherein the upper and lower gripping surfaces are at least partially convex.

4. The spacer of claim 3, wherein the upper and lower gripping surfaces are recessed with respect to the upper and lower bone engaging surfaces.

5. The spacer of claim 4, wherein the upper and lower gripping surfaces are roughened.

6. The spacer of claim 1, wherein the spacer further includes a plurality of tool engagement configurations adapted for engagement by an insertion tool over a range of angles with respect to a longitudinal axis of the insertion tool.

7. The spacer of claim 1, wherein the upper and lower engaging surfaces are load bearing surfaces.

8. The spacer of claim 1, wherein the upper and lower engaging surfaces include a plurality of projections.

9. The spacer of claim 1, wherein the spacer is formed of a material suitable for human implantation.

10. The spacer of claim 9, wherein the spacer is formed of a substantially solid material.

11. The spacer of claim 9, wherein the material is bone.

12. The spacer of claim 9, wherein the material is synthetic.

13. The spacer of claim 3, wherein the upper and lower convex surfaces are arcuate surfaces.

14. The spacer of claim 13, wherein the upper and lower arcuate surfaces have substantially the same radius of curvature.

15. The spacer of claim 14, wherein the spacer has a height between the upper and lower bone engaging surfaces and the radius of curvature forms an arc having a diameter that is greater than the height.

16. The spacer of claim 14, wherein the spacer has a height between the upper and lower bone engaging surfaces and the radius of curvature forms an arc having a diameter that is less than the height.

17. The spacer of claim 3, wherein the gripping surfaces from at least a partially cylindrical surface.

18. The spacer of claim 17, wherein the upper and lower gripping surfaces are spaced from each other by an exterior sidewall having a first height.

19. The spacer of claim 18, wherein the spacer has an interior area and further includes an interior sidewall extending between the upper and lower gripping surfaces, the interior sidewall having a second height.

20. The spacer of claim 19, wherein the first height is substantially equal to the second height.

21. The spacer of claim 19, wherein the first height is greater than the second height.

22. The spacer of claim 19, wherein the first height is less than the second height.

23. The spacer of claim 1, wherein the tool engagement configuration extends at least partially transverse to the spacer axis.

24. The spacer of claim 23, wherein the tool engagement configuration has a curved shape within the horizontal plane.

25. The spacer of claim 1, wherein the spacer has an spacer height extending between the upper and lower bone engaging surfaces and the tool engagement configuration has a gripping height extending between the upper and lower gripping surfaces, the gripping height being less than the spacer height.

26. The spacer of claim 25, wherein the tool engagement configuration is a projection extending at least partially beyond the perimeter of the upper and lower bone engaging surfaces.

27. The spacer of claim 26, wherein the projection is at least partially cylindrical.

28. The spacer of claim 26, wherein the projection is at least partially spherical.

29. The spacer of claim 1, wherein the insertion tool is positionable at an infinite number of angular orientations.

30. The spacer of claim 1, wherein the insertion tool is positionable at a plurality of discrete angular orientations.

31. The spacer of claim 30, wherein the upper and lower gripping surfaces include a plurality of angularly spaced recesses adapted to receive a projection of the insertion tool.

32. The spacer of claim 1, wherein the spacer is a corpectomy device.

33. The spacer of claim 1, wherein the spacer is a nucleus replacement.

34. The spacer of claim 1, wherein the spacer is an artificial disc.

35. A multi-grip insertion tool for holding a spacer for insertion at least partially between two vertebrae, the spacer having an spacer axis, the insertion tool comprising:
a shaft having a proximal portion and a distal portion;
a spacer gripping end disposed adjacent the distal portion having a longitudinal axis, the spacer gripping end having an upper portion and a lower portion, the upper portion having a first concave inner surface, the lower portion having a second concave inner surface, the first inner surface oriented with the second inner surface so as to facilitate engagement of the spacer at a plurality of angular orientations between the longitudinal axis and the spacer axis.

36. The insertion tool of claim 35, wherein the first and second concave inner surfaces are arcuate surfaces.

37. The insertion tool of claim 36, wherein the first and second arcuate surfaces have substantially the same radius of curvature.

38. The insertion tool of claim 37, wherein the spacer gripping end has a channel height between the upper portion and the lower portion and the radius of curvature forms an arc having a diameter that is greater than the channel height.

39. The insertion tool of claim 37, wherein the spacer gripping end has a channel height between the upper portion and the lower portion and the radius of curvature forms an arc having a diameter that is less than the channel height.

40. The insertion tool of claim 35, wherein the first inner surface includes a projection adapted for engaging a recess of the spacer.

41. The insertion tool of claim 40, wherein the second inner surface includes a projection adapted for engaging a recess of the spacer.

42. The insertion tool of claim 35, wherein the orientation of the first and second inner surfaces forms a substantially cylindrical cavity.

43. The insertion tool of claim 35, wherein the first inner surface includes a first proximal edge and a first distal edge, the first proximal edge being concave and the first distal edge being convex.

44. The insertion tool of claim 43, wherein the first proximal edge and the first distal edge are arcuate.

45. The insertion tool of claim 44, wherein the arcuate edges have substantially the same radius of curvature.

46. The insertion tool of claim 44, wherein the proximal edge has a greater radius of curvature than the distal edge.

47. The insertion tool of claim 43, wherein the second inner surface includes a second proximal edge and a second distal edge, the second proximal edge being concave and the second distal edge being convex.

48. The insertion tool of claim 47, wherein the first and second proximal edges are arcuate and the first and second distal edges are arcuate.

49. The insertion tool of claim 48, wherein the arcuate proximal edges and the arcuate distal edges have substantially the same radius of curvature.

50. The insertion tool of claim 48, wherein the arcuate proximal edges have substantially the same radius of curvature, the arcuate distal edges have substantially the same radius of curvature, and the arcuate proximal edges have a radius of curvature that is greater than the radius of curvature of the arcuate distal edges.

51. The insertion tool of claim 35, wherein the first inner surface is at least partially spherical.

52. The insertion tool of claim 51, wherein the second inner surface is at least partially spherical.

53. The insertion tool of claim 52, wherein the orientation of the first inner surface to the second inner surface forms a substantially spherical cavity.

54. The insertion tool of claim 35, wherein the insertion tool is positionable at an infinite number of angular orientations for engaging the spacer.

55. The insertion tool of claim 35, wherein the insertion tool is positionable at a plurality of discrete angular orientations for engaging the spacer.

56. A multi-grip insertion tool for holding a spacer for insertion at least partially into a disc space between two adjacent vertebrae, the spacer having an spacer axis, the insertion tool comprising:
a shaft having a proximal portion and a distal portion;
an spacer gripping end disposed adjacent the distal portion having a longitudinal axis; and
a means for selectively engaging a spacer at a plurality of angles between the longitudinal axis and the spacer axis.

57. The insertion tool of claim 56, wherein the means for selectively engaging the spacer includes engaging an exterior surface of the spacer.

58. The insertion tool of claim 57, wherein the exterior surface of the spacer is substantially convex.

59. The insertion tool of claim 56, wherein the means for selectively engaging the spacer includes a movably engaged position.

60. The insertion tool of claim 59, wherein the movably engaged position allows rotational movement of the spacer with respect to the insertion tool.

61. The insertion tool of claim 59, wherein the movably engaged position allows translational movement of the spacer with respect to the insertion tool.

62. The insertion tool of claim 56, wherein the means for selectively engaging the spacer includes engaging a cavity of the spacer.

63. The insertion tool of claim 62, wherein the gripping end further includes an expandable projection and the means for selectively engaging the spacer includes expanding the expandable projection.

64. A multi-grip insertion tool for holding a spacer for insertion at least partially between two vertebrae, the spacer having an spacer axis, the insertion tool comprising:
a shaft having a proximal portion and a distal portion;
a movable gripping end disposed adjacent the distal portion having a longitudinal axis, the movable gripping end having an upper portion and a lower portion, the upper portion having a first concave inner surface, the lower portion having a second concave inner surface, the first inner surface oriented with the second inner surface so as to facilitate engagement of the spacer at a plurality of angular orientations between the longitudinal axis and the spacer axis, the movable gripping end includes an open position configured to allow passage of the spacer, a movably engaged position configured to retain the spacer yet allowing motion between the spacer and the gripping end, and a locked position configured to prevent movement of the spacer with respect to the gripping end; and
an actuator disposed adjacent the proximal portion, the actuator adapted for selectively moving the movable gripping end between the open, movably engaged, and locked position.

65. The insertion tool of claim 64, wherein the movably engaged position permits rotational movement of the spacer with respect to the insertion tool.

66. The insertion tool of claim 64, wherein the movably engaged position permits translational movement of the spacer with respect to the insertion tool.

67. The insertion tool of claim 65, wherein the movably engaged position permits translational movement of the spacer with respect to the insertion tool.

68. The insertion tool of claim 67, wherein the first and second concave inner surfaces are arcuate surfaces.

69. The insertion tool of claim 68, wherein the first and second arcuate surfaces have substantially the same radius of curvature.

70. The insertion tool of claim 69, wherein the spacer gripping end has a channel height between the upper portion and the lower portion and the radius of curvature forms an arc having a diameter that is greater than the channel height.

71. The insertion tool of claim 69, wherein the spacer gripping end has a channel height between the upper portion and the lower portion and the radius of curvature forms an arc having a diameter that is less than the channel height.

72. The insertion tool of claim 64, wherein the first inner surface includes a first proximal edge and a first distal edge, the second inner surface includes a second proximal edge and a second distal edge, the first and second proximal edges being concave and the first and second distal edges being convex.

73. The insertion tool of claim 72, wherein the first and second proximal edges are arcuate and the first and second distal edges are arcuate.

74. The insertion tool of claim 73, wherein the arcuate proximal edges and the arcuate distal edges have substantially the same radius of curvature.

75. The insertion tool of claim 73, wherein the arcuate proximal edges have substantially the same radius of curvature, the arcuate distal edges have substantially the same radius of curvature, and the arcuate proximal edges have a radius of curvature that is greater than the radius of curvature of the arcuate distal edges.

76. The insertion tool of claim 64, wherein the orientation of the first and second inner surfaces forms a substantially cylindrical cavity.

77. The insertion tool of claim 64, wherein the insertion tool is positionable at an infinite number of angular orientations for engaging the spacer.

78. The insertion tool of claim 64, wherein the insertion tool is positionable at a plurality of discrete angular orientations for engaging the spacer.

79. A combination spacer for insertion at least partially into the disc space between two adjacent vertebrae and a multi-grip insertion tool for gripping the spacer, the combination comprising:
a spacer body having a first multi-angle engagement area and an spacer axis;
an insertion tool with a shaft having a distal portion and a proximal portion;
a spacer gripping end disposed adjacent to the distal portion and having a longitudinal axis, the spacer gripping end configured for gripping the first multi-angle engagement area at a plurality of angles between the longitudinal axis and the spacer axis.

80. The combination of claim 79, wherein the spacer body includes a plurality of multi-angle engagement areas and the spacer gripping end is configured for gripping the spacer via any of the plurality of multi-angle engagement areas at a plurality of angles with respect to the longitudinal axis.

81. The combination of claim 79, wherein the first multi-angle engagement area includes a concave portion and the spacer gripping end has a corresponding convex portion.

82. The combination of claim 79, wherein the first multi-angle engagement area includes a convex portion and the spacer gripping end has a corresponding concave portion.

83. The combination of claim 82, wherein the spacer includes an upper convex surface, a lower convex surface, and a horizontal plane between the upper and lower convex surfaces.

84. The combination of claim 83, wherein the plurality of engagement angles are within the horizontal plane.

85. The combination of claim 83, wherein the plurality of engagement angles are oblique to the horizontal plane.

86. The combination of claim 83, wherein the plurality of engagement angles includes angles within and oblique to the horizontal plane.
